(19) United States
(12) Patent Application Publication
(10) Pub. No.: US 2013/0172570 A1
(43) Pub. Date: Jul. 4, 2013

(54) METHOD OF MAKING BENZAZOLES

(71) Applicants: Jianmin Shi, Rockville, MD (US); Eric W. Forsythe, Rockville, MD (US); David C. Morton, Columbia, MD (US)

(72) Inventors: Jianmin Shi, Rockville, MD (US); Eric W. Forsythe, Rockville, MD (US); David C. Morton, Columbia, MD (US)

(73) Assignee: US Government as Represented by the Secretary of the ARMY, Adelphi, MD (US)

(21) Appl. No.: 13/396,805

(22) Filed: Feb. 15, 2012

Related U.S. Application Data
(60) Provisional application No. 61/581,355, filed on Dec. 29, 2011.

(51) Int. Cl. C07D 403/06; C07D 403/14; C07D 403/08; C07D 235/18; C07D 263/56; C07D 277/64

(52) U.S. Cl. 548/152; 548/305.7; 548/224; 548/305.4; 548/310.7

(57) ABSTRACT

This present invention provides a method of making benzazoles comprising a process of making aryl or alkyl benzazoles from corresponding aryl acid chlorides or alkyl acid chlorides without applying hazard condensing agent. The benzazole compounds described in this invention have following formula I:

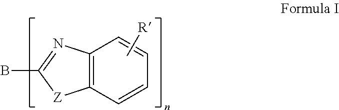

Wherein:
n is an integer of from 3 to 8;
Z is O, NR or S; and
R and R′ are individually hydrogen; alkyl of from 1 to 24 carbon atoms, for example, propyl, t-butyl, heptyl, and the like; aryl or hetero-atom substituted aryl of from 5 to 20 carbon atoms, for example, phenyl and naphthyl, furyl, thienyl, pyridyl, quinolinyl and other heterocyclic systems; or halo such as chloro, fluoro, bromo, cyano; or atoms necessary to complete a fused aromatic ring; and
B is a linkage unit consisting of alkyl, aryl, substituted alkyl, or substituted aryl which conjugately or unconjugately connects the multiple benzazoles together.

### BENEFIT OF PRIORITY

Applicants hereby claim benefit of U.S. Provisional Application 61/581,355 filed 29 Dec. 2011.

### GOVERNMENT INTEREST

The invention described herein may be manufactured, used, and licensed by or for the United State Government.

### FIELD OF THE INVENTION

This invention relates to a novel method of making benzazoles (BZZ). Specifically, this method is related to a one step process of making aryl or alkyl benzazoles from the corresponding aryl acid chlorides or alkyl acid chlorides.

### BACKGROUND OF THE INVENTION

Benzazole derivatives, typically aryl benzazole derivatives, have been widely used for many applications. More typically these benzazoles are used as surface coatings for absorbers of UV light. In addition, they are used in organic electroluminescent (EL) devices. In electroluminescent devices they are known to be highly efficient and capable of producing a wide range of colors. Also, they are significantly important for energy efficient solid state lighting as well as full color flat-panel displays.

In U.S. Pat. Nos. 5,645,948 and 5,766,779, Shi et. al. discloses organic electroluminescent (EL) elements that belong to benzazoles derivatives, more typically aryl benzoimidazoles. These aryl benzoimidazoles provide a thermally stable and highly fluorescent material as condensed thin films which dramatically exhibit blue emission organic EL performance. As a result, organic EL devices employing these benzoimidazoles derivatives, more typically, 2,2′,2″-(1,3,5-phenylene)tris[1-phenyl-1H-benzimidazole] (TPBI), in a light-emitting layer produce a bright blue emission with long operational stability. Further, these benzoimidazole derivatives are used as an electron transport layer with great electron injection properties. In recent years, these benzoimidazoles are more and more widely used in organic EL devices as a hole-blocking layer.

Shi's original U.S. Pat. Nos. 5,645,948 and 5,766,779 use a two step synthesis of benzazoles derivatives from aryl acid chlorides by high temperature condensation. After being separated, the corresponding amide has been found.

Recently, Guidry et al. of E. I. du Pont de Nemours and Company disclosed another two step synthesis of this class of material from acid chlorides by condensing the amide with a proximate anilino group present in the adduct in the presence of the condensing agent (see U.S. Pat. No. 7,273,939). In contrast, the present invention, discloses a novel method of making benzimidazoles in a one step process of making aryl or alkyl benzimidazole derivatives from the corresponding aryl acid chlorides or alkyl acid chlorides without applying condensing agent.

### SUMMARY OF THE INVENTION

It is the object of this present invention to provide a method of making benzazoles. More specifically, it is an object of this invention to provide a one step process of making aryl or alkyl benzazoles from the corresponding aryl acid chlorides or alkyl acid chlorides without applying hazardous condensing agents. The benzazole compounds described in this invention have the following formula I:

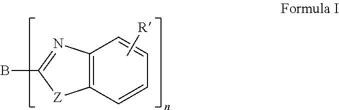

Wherein:
n is an integer of from 3 to 8;
Z is O, NR or S; and
R and R′ are individually hydrogen; alkyl of from 1 to 24 carbon atoms, for example, propyl, t-butyl, heptyl, and the like; aryl or hetero-atom substituted aryl of from 5 to 20 carbon atoms, for example, phenyl and naphthyl, furyl, thienyl, pyridyl, quinolinyl and other heterocyclic systems; or halo such as chloro, fluoro, bromo, cyano; or atoms necessary to complete a fused aromatic ring; and
B is a linkage unit consisting of alkyl, aryl, substituted alkyl, or substituted aryl which conjugately or unconjugately connects the multiple benzazoles together.

### DETAILED DESCRIPTION OF THE PREFERRED EMBODIMENTS

The general method of making benzazoles of present invention can be described in scheme 1 as followings:

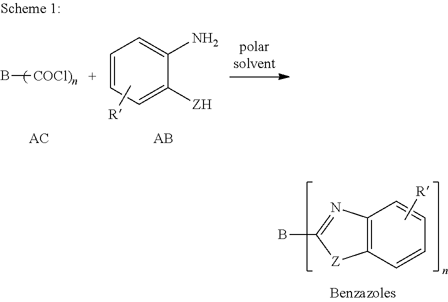

In a typical example, 2-aminobenzols (AB), aryl or alkyl acid chloride (AC) was slowly added to a polar solvent under moisture protection. Preferably, the polar solvent, such as N-methyl-2-pyrrolidinone (NMP), has a high boiling temperature. The reaction mixture was stirred at ambient temperature from one to several hours under moisture protection. Then, with vigorous stirring, the reaction mixture was raised to a higher temperature (up to 205° C.) for a few hours until the reaction was completed. The reaction mixture was then cooled to room temperature and poured into water with vigorous stirring. The resultant precipitates were filtered and washed with a carefully chosen solvent, such as water, methanol, ethanol, acetone or mixture of them. In most cases, the product can be directly sublimed for organic EL device fabrication without going through future purification process.

In 2-aminobenzols (AB), Z is O, NR or S; and R and R′ are individually hydrogen; alkyl of from 1 to 24 carbon atoms, for example, propyl, t-butyl, heptyl, and the like; aryl or hetero-atom substituted aryl of from 5 to 20 carbon atoms, for example, phenyl and naphthyl, furyl, thienyl, pyridyl, quinolinyl and other heterocyclic systems; or halo such as chloro, fluoro, bromo, cyano; or atoms necessary to complete a fused aromatic ring.

The more typical 2-aminobenzols (AB) are selected from but not limited to the following list:

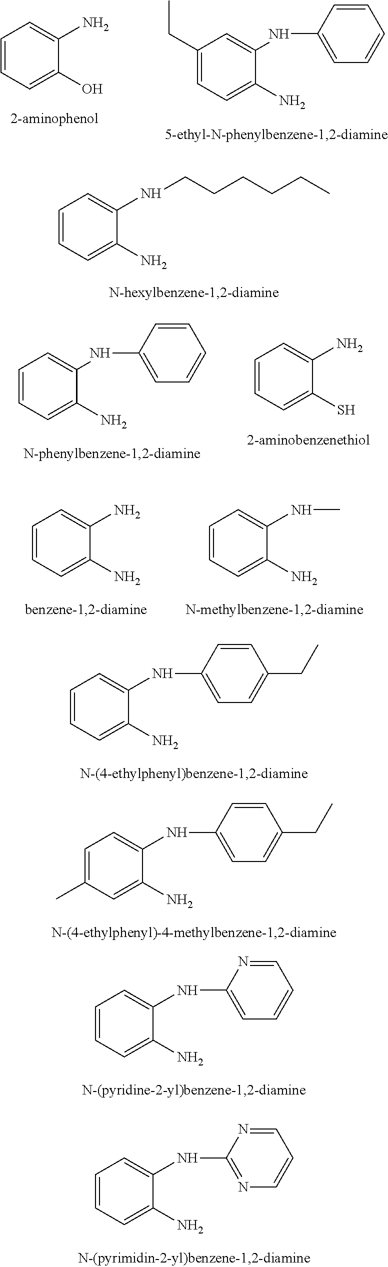

For aryl or alkyl acid chloride (AC), B is a linkage unit consisting of alkyl, aryl, substituted alkyl, or substituted aryl which conjugately or unconjugately connects the multiple acid chlorides together.

The aryl or alkyl acid chloride (AC) are selected from the following list but not limited to the list:

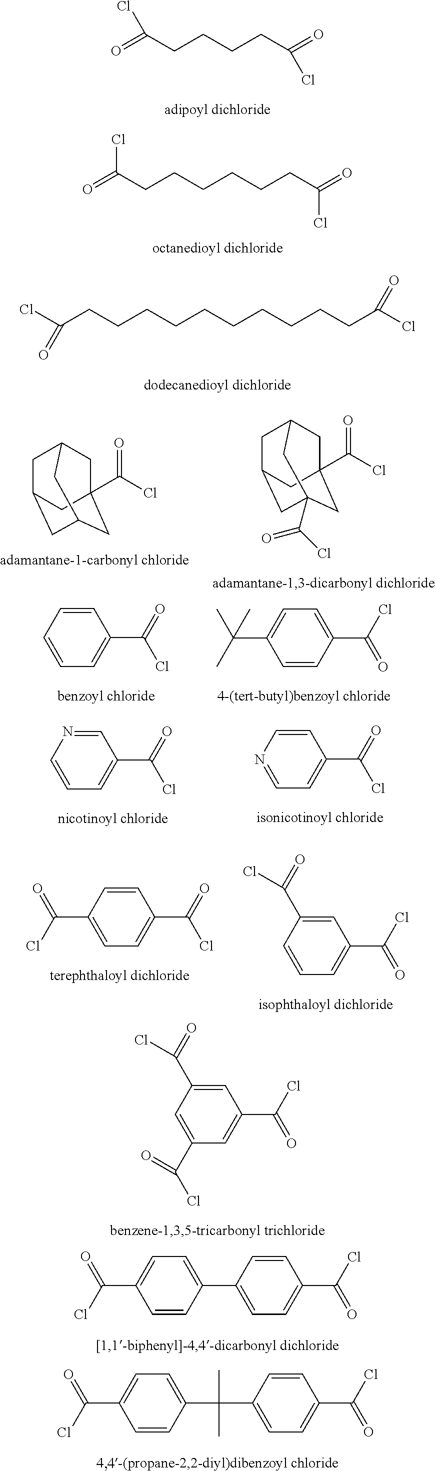

The preferred polar solvents contain at least an amino group which helps to take up generated hydrogen chloride from the reaction. In addition, solvents with high boiling temperature are preferred because they help condensation carried out at atmospheric conditions. The more suitable polar solvents are selected from the following list but not limited to the list:

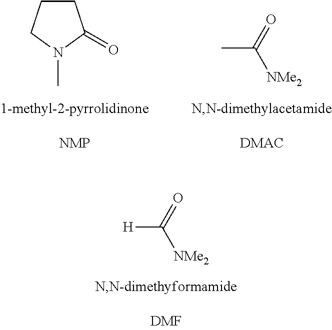

The following benzazoles (BZZ) are listed, but not limited, as examples made by the method of the present invention.

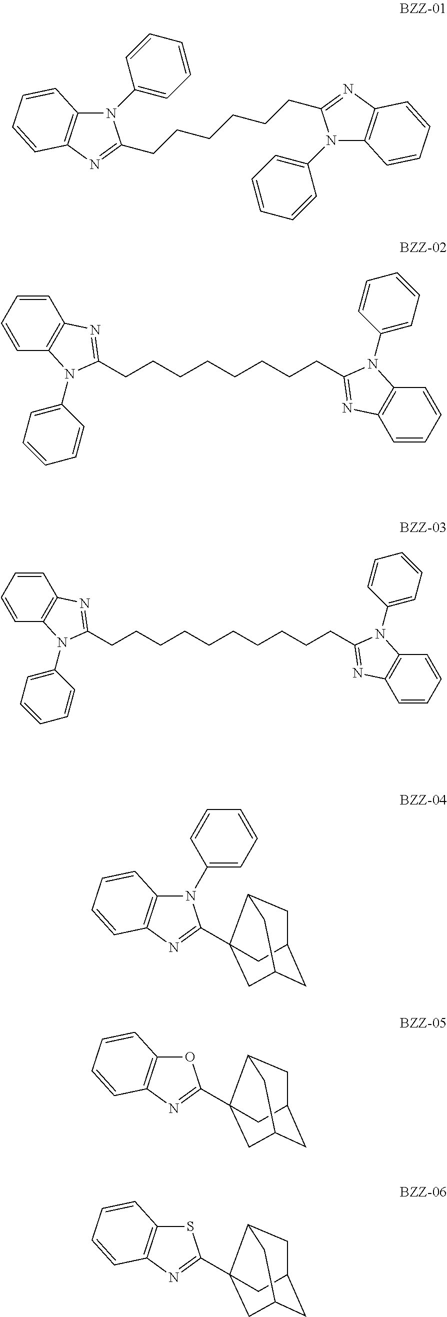 [SMILES: c1ccc(-n2c(CCCCCCc3nc4ccccc4n3-c3ccccc3)nc3ccccc32)cc1 ; c1ccc(-n2c(CCCCCCCCc3nc4ccccc4n3-c3ccccc3)nc3ccccc32)cc1 ; c1ccc(-n2c(CCCCCCCCCCc3nc4ccccc4n3-c3ccccc3)nc3ccccc32)cc1 ; c1ccc(-n2c(C34CC5CC(CC3C5)C4)nc3ccccc32)cc1 ; c1ccc2oc(C34CC5CC(CC3C5)C4)nc2c1 ; c1ccc2sc(C34CC5CC(CC3C5)C4)nc2c1]

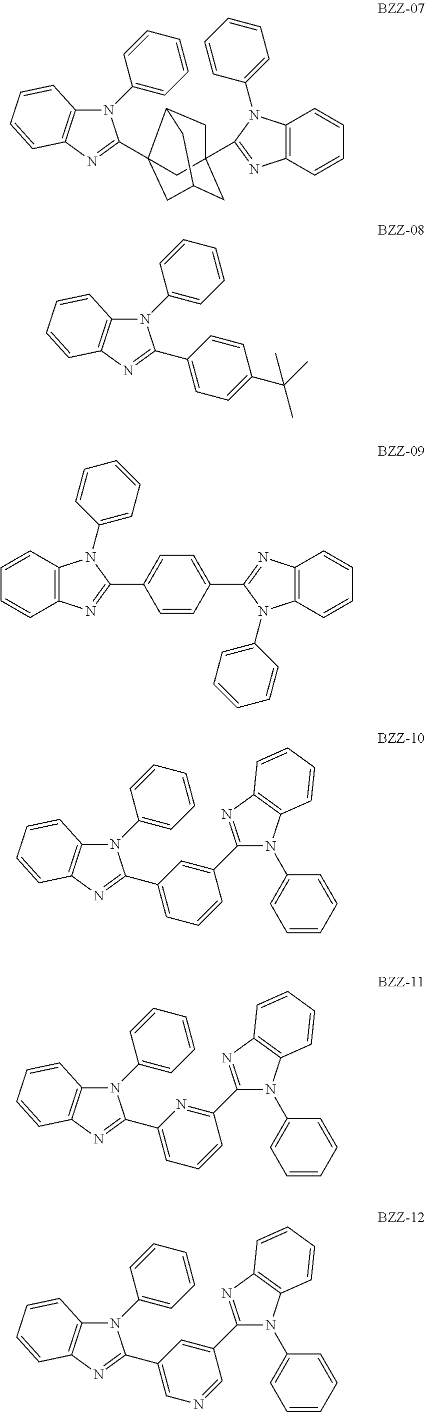

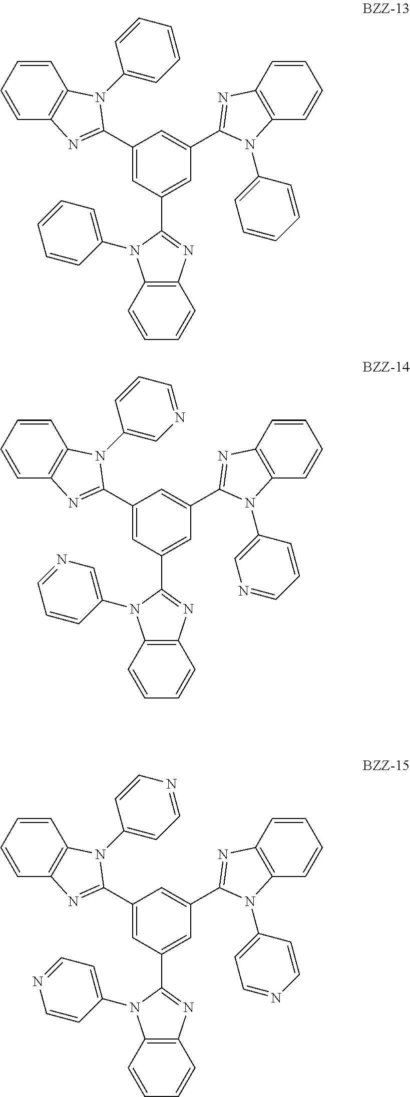

The following examples illustrate how the above described general procedure of the invention is carried out.

### EXAMPLES

### Example 1

### Synthesis of 1,6-bis-(2-(1-phenylbenzimidazole) hexane (BZZ-01)

Suberoyl chloride (5.0 mL, 0.028 mol), N-phenyl-o-phenylenediamine (10.2 g, 0.055 mol) and anhydrous N-Methylpyrrolidinone (40 mL) were combined and heated to 80° C. for 1 hour and then at 170° C.-180° C. for 2 hours. The reaction mixture was cooled, added to a separatory funnel containing water (200 mL) and dichloromethane (200 mL) and shook. The aqueous layer was removed and the organic layer was washed twice more with water and then finally with brine. The organic layer was removed and concentrated by evaporation. Addition of ethyl acetate and further concentration led to the precipitation of the product, which was filtered off. The product was further purified by re-crystallization from methanol and ethyl acetate. Filtration and oven drying (90° C.) resulted in 9.46 g of an off white powder (73%). MS: m/z=471.0 (M+H⁺)

### Example 2

### Synthesis of 1,8-bis-(2-(1-phenylbenzimidazole) octane (BZZ-02)

Sebacoyl chloride (5.8 mL, 0.027 mol), N-phenyl-o-phenylenediamine (10.1 g, 0.055 mol) and anhydrous N-Methylpyrrolidinone (35 mL) were combined and heated to 80° C. for 1 hour and then at 170° C.-180° C. for 2 hours. The reaction mixture was cooled, added to a separatory funnel containing water (200 mL) and dichloromethane (200 mL) and shook. The aqueous layer was removed and the organic layer was washed twice more with water and then finally with brine. The organic layer was removed and concentrated by evaporation. Addition of ethyl acetate and further concentration led to the precipitation of the product, which was filtered off. The product was further purified by re-crystallization from methanol and ethyl acetate. Filtration and oven drying (90° C.) resulted in 6.15 g of an off white powder (45%). MS: m/z=499.0 (M+H⁺)

### Example 3

### Synthesis of 1,10-bis-(2-(1-phenylbenzimidazole) decane (BZZ-03)

Dodecanedioyl dichloride chloride (6.0 mL, 0.024 mol), N-phenyl-o-phenylenediamine (8.84 g, 0.054 mol) and anhydrous N-Methylpyrrolidinone (35 mL) were combined and heated to 80° C. for 1 hour and then at 170° C.-180° C. for 2 hours. The reaction mixture was cooled, added to a separatory funnel containing water (200 mL) and dichloromethane (200 mL) and shook. The aqueous layer was removed and the organic layer was washed twice more with water and then finally with brine. The organic layer was removed and concentrated by evaporation. Addition of ethyl acetate and further concentration led to the precipitation of the product, which was filtered off. The product was further purified by re-crystallization from methanol and ethyl acetate. Filtration and oven drying (90° C.) resulted in 4.0 g of an off white powder (32%). MS: m/z=527.1 (M+H⁺)

### Example 4

### Synthesis of 2-(1-adamantane)-benzoxazole (BZZ-05)

2-aminophenol (1.82 g, 0.017 mol), 1-adamantanecarbonyl chloride (3.50 g, 0.018 mol) and N-Methyl-2-pyrrolidinone (20 mL) were combined and heated to 80° C. for 1 hour and then at 170° C.-180° C. for 2 hours. After cooling, water (100 mL) was added and the mixture was sonicated to induce precipitation. The product was filtered, re-dissolved in dichloromethane and dried over Na₂SO₄. The solvent was removed under reduced pressure and the residue was chromatographed on silica gel using 1:1 dichloromethane:hexanes for eluent. Concentration led to the precipitation of the product, which was collected and dried in vacuum (1.7 g yield). MS: m/z=254.1 (M+H⁺)

### Example 5

### Synthesis of 2-(1-adamantane)-benzothiazole (BZZ-06)

2-aminothiophenol (2.0 mL, 0.019 mol), 1-adamantanecarbonyl chloride (3.67 g, 0.018 mol) and N-Methylpyrrolidinone (20 mL) were combined and heated to 80° C. for 1 hour and then at 170° C.-180° C. for 2 hours. After cooling, water (100 mL) was added and the mixture was sonicated to induce precipitation. The product was filtered, re-dissolved in dichloromethane and dried over Na₂SO₄. The solvent was removed under reduced pressure and the residue was chromatographed on silica gel using 1:1 dichloromethane:hexanes for eluent. Concentration led to the precipitation of the product, which was collected and dried in vacuum (1.6 g yield). MS: m/z=270.0 (M+H⁺)

### Example 6

### Synthesis of 3,5-bis-2-(1-phenylbenzimidazole)-tert-butylbenzene (BZZ-07)

5-tert-butylisophthalic acid (15 g, 0.067 mol) and thionyl chloride (50 mL, 0.68 mol) were combined and refluxed for 4 h. Most of the thionyl chloride was distilled off under reduced pressure and dry hexanes (50 mL) were added to the residue. The resulting white precipitate was filtered and washed with additional dry hexanes (50 mL). The acid chloride was transferred to another flask which contained N-phenyl-o-phenylenediamine (21.5 g, 0.117 mol) and anhydrous N-Methylpyrrolidinone (75 mL). The solution was heated to 80° C. for 1 h then to 170° C.˜180° C. for 2 hours. Afterwards, the reaction mixture was cooled and water (250 mL) was added to induce precipitation of the product, which was filtered off. The product was re-dissolved in dichloromethane and dried by shaking with brine. The organic layer was removed and concentrated by evaporation. Addition of ethyl acetate and further evaporation resulted in precipitation of the product which was filtered, dried and purified by sublimation. MS: m/z=518.9 (M+H⁺)

### Example 7

### Synthesis of 1-phenyl-2-(4-tert-butylphenyl)benzimidazole (BZZ-08)

4-tert-butylbenzoyl chloride (5.0 mL, 0.028 mol), N-phenyl-o-phenylenediamine (5.1 g, 0.028 mol) and anhydrous N-Methylpyrrolidinone (20 mL) were combined and heated to 80° C. for 1 hour and then at 170° C.-180° C. for 2 hours. The reaction mixture was cooled, added to a separate funnel containing water (150 mL) and dichloromethane (150 mL) and shook. The aqueous layer was removed and the organic layer was washed twice more with water and then finally with brine. The organic layer was removed and concentrated by evaporation (recovered 8.5 g crude). The crude product was chromatographed on silica gel using 5% MeOH in dichloromethane for eluent. Concentration by evaporation and addition of ethyl acetate resulted in precipitation of the product, which was filtered and dried in vacuum. MS: m/z=327.1 (M+H⁺)

### Example 8

### Synthesis of 1,3,5-tris(1-phenyl-1H-benzo[d]imidazol-2-yl)benzene (BZZ-13) [TPBI]

Benzene-tricarbonyl trichloride (7.4 g, 0.028 mol.) was slowly added to 50 mL of anhydrous N-Methylpyrrolidinone (NMP) containing 19.5 g (0.106 mol.) of N-phenyl-o-phenylenediamine under moisture protection. Then the reaction mixture was heated from room temperature to 80° C. under stirring for 1 hour and then raises the reaction temperature to 180° C.-195° C. for 6 hours. After the reaction mixture was cooled to room temperature, 200 mL of water was added to reaction mixture with vigorously stirring. Then resulted precipitates were filtered and washed with water, mixture of water/alcohol. The pure product of 1,3,5-tris(1-phenyl-1H-benzo[d]imidazol-2-yl)benzene (BZZ-13) [TPBI] (15.1 g) was obtained at yield of 82.5% by finally washed with acetone and dried.

With the invention having been described in general and in detail and the reference to specific embodiments thereof, it will be apparent to one ordinarily skilled in the art that various changes, alterations, and modifications can be made without departing from the spirit and scope of the invention and its equivalents as defined by the appended claims.

What is claimed is:

1. A method of making benzazole compounds of formula I: 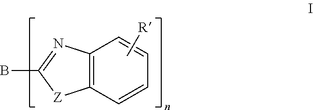
Wherein:
n is an integer of from 3 to 8;
Z is O, NR or S; and
R and R′ are individually hydrogen; alkyl of from 1 to 24 carbon atoms and the like; aryl or hetero-atom substituted aryl of from 5 to 20 carbon atoms and other heterocyclic systems; or halo selected from the group consisting of chloro, fluor bromo; or cyano; or atoms necessary to complete a fused aromatic ring; and
B is a linkage unit consisting of alkyl, aryl, substituted alkyl, or substituted aryl which conjugately or unconjugately connects the multiple benzazoles together by a one step process of

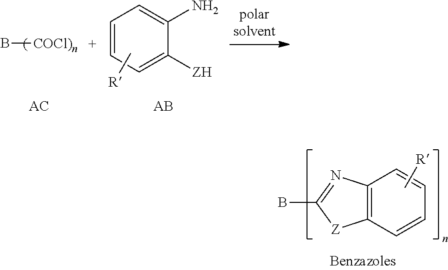

2. The method according claim 1, wherein the benzazoles are prepared by one step process from corresponding aryl acid chlorides or alkyl acid chlorides (AC) and 2-aminobenzols (AB) in a polar solvent.

3. The method according claim 2, wherein in the structure of 2-aminobenzols (AB) Z is O, NR or S; and R and R′ are individually hydrogen; alkyl of from 1 to 24 carbon atoms, aryl or hetero-atom substituted aryl of from 5 to 20 carbon atoms, or halo such as chloro, fluoro, bromo, cyano; or atoms necessary to complete a fused aromatic ring.

4. The method according claim 3, wherein the 2-aminobenzols is N-phenylbenzene-1,2-diamine of the formula:

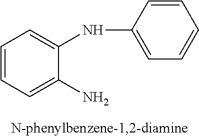 [SMILES: Nc1ccccc1Nc1ccccc1]

5. The method according claim 3, wherein the structure of the aryl or alkyl acid chloride (AC) B is a linkage unit consisting of alkyl, aryl, substituted alkyl, or substituted aryl which conjugately or unconjugately connects the multiple acid chlorides together.

6. The method according claim 5, wherein the aryl acid chloride is benzene-1,3,5-tricarbonyl chloride.

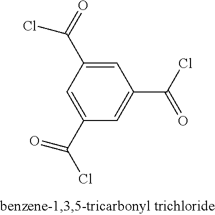 [SMILES: O=C(Cl)c1cc(C(=O)Cl)cc(C(=O)Cl)c1]

7. The method according claim 3, wherein the boiling temperature of polar solvent is higher than 120° C.

8. The method according claim 7, wherein the polar solvent is 1-methyl-2-pyrrolidinone (NMP), N,N-dimethylacetamide (DMAC) and N,N-dimethylformamide (DMF); or combinations thereof.

9. The method according claim 1, wherein benzazole is 1,10-bis(1-phenyl-1H-benzo[d]imidazol-2-yl)decane

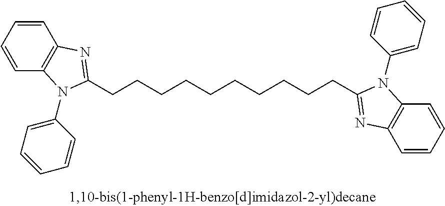 [SMILES: c1ccc(-n2c(CCCCCCCCCCc3nc4ccccc4n3-c3ccccc3)nc3ccccc32)cc1]

10. The method according claim 1, wherein benzazole is 1,3,5-tris(1-phenyl-1H-benzo[d]imidazol-2-yl)benzene. 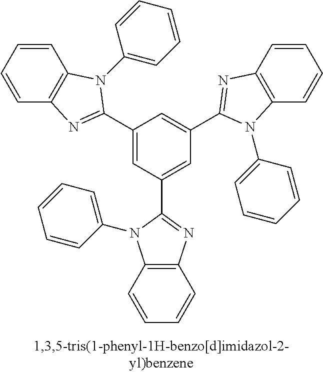 [SMILES: c1ccc(-n2c(-c3cc(-c4nc5ccccc5n4-c4ccccc4)cc(-c4nc5ccccc5n4-c4ccccc4)c3)nc3ccccc32)cc1]
